(19) United States
(12) Patent Application Publication
(10) Pub. No.: US 2025/0151963 A1
(43) Pub. Date: May 15, 2025

(54) CLEANER STATION AND CLEANING DEVICE WITH STERILIZING MODULE

(71) Applicant: Samsung Electronics Co., Ltd., Suwon-si (KR)

(72) Inventor: Donghyun LEE, Suwon-si (KR)

(73) Assignee: Samsung Electronics Co., Ltd., Suwon-si (KR)

(21) Appl. No.: 19/022,024

(22) Filed: Jan. 15, 2025

Related U.S. Application Data
(63) Continuation of application No. PCT/KR2023/012266, filed on Aug. 18, 2023.

(30) Foreign Application Priority Data
Oct. 11, 2022 (KR) 10-2022-0129758

(51) Int. Cl. A47L 7/00; A47L 9/00; A47L 9/14; A47L 9/28; A61L 2/10; A61L 2/24

(52) U.S. Cl. CPC A47L 7/0061; A47L 9/0063; A47L 9/1427; A47L 9/149; A47L 9/2805; A47L 9/2836; A61L 2/10; A61L 2/24; A61L 2202/11; A61L 2202/121; A61L 2202/14; A61L 2202/16; A61L 2202/17; A61L 2202/23

(57) ABSTRACT

According to an embodiment of the present disclosure, a cleaner station includes a sterilization module arranged inside a main body and configured to emit ultraviolet rays into a dust bag, wherein the sterilization module includes an ultraviolet unit configured to emit ultraviolet rays into the main body, and a blocking member to selectively block the ultraviolet rays emitted by the ultraviolet unit, wherein the blocking member moves to a blocking position to block the ultraviolet rays emitted by the ultraviolet unit from entering the main body, in conjunction with a positional movement of the dust bag, when the dust bag is detached from the main body, and moves to an open position to allow the ultraviolet rays emitted by the ultraviolet unit to enter the main body, in conjunction with a positional movement of the dust bag, when the dust bag is mounted on the main body.

### CROSS-REFERENCE TO RELATED APPLICATIONS

This application is a continuation of International Application PCT/KR2023/012266, filed Aug. 18, 2023, and claims foreign priority to Korean Application 10-2022-0129758, fled Oct. 11, 2022, and which are incorporated herein by reference in their entireties.

### TECHNICAL FIELD

Embodiments provide a cleaner station having a sterilization module, and a cleaning device.

### BACKGROUND ART

Vacuum cleaners are electronic devices for sucking in air containing foreign substances such as dust by using negative pressure, and then filtering out, inside a main body, the foreign substances. Such vacuum cleaners may include a dust collection container that separates and collects dust from the sucked-in air.

The separated foreign substances are collected into the dust collection container, and the collected foreign substances need to be removed periodically. However, it may be cumbersome for a user to remove the foreign substances from the dust collection container, and during a process of removing the foreign substances, dust may get scattered again, increasing the indoor dust concentration.

In order to reduce the inconvenience experienced by a user when manually removing foreign substances, a cleaner station may be connected to a vacuum cleaner to automatically discharge foreign substances inside a dust collection container.

### DISCLOSURE OF INVENTION

### Solution to Problem

A cleaner station according to an embodiment may include a main body on which a dust bag is detachably mountable to collect foreign substances in the dust bag, and a sterilization module.

The sterilization module may include an ultraviolet unit configured to emit ultraviolet rays into the main body, and a blocking member.

The blocking member may be configured to move to (a) a blocking position to block the ultraviolet rays emitted by the ultraviolet unit from entering the main body, in conjunction with a positional movement of the dust bag, when the dust bag is detached from the main body, and (b) an open position so that the ultraviolet rays emitted by the ultraviolet unit enter the main body and the dust bag, in conjunction with a positional movement of the dust bag, when the dust bag is mounted on the main body.

A cleaning device according to an embodiment may include a vacuum cleaner including a dust collection container in which foreign substances are collectable, and a cleaner station.

The cleaner station may include a docking unit to which the dust collection container is dockable, a main body to which a dust bag is detachably mountable, a suction unit, and a sterilization module.

The suction unit may be configured to provide a suction force to, with the dust collection container docked to the docking unit and the dust bag mounted to the main body, cause foreign substances collected in the dust collection container to move to the dust bag.

The sterilization module may include an ultraviolet unit configured to emit ultraviolet rays into the main body, and a blocking member.

The blocking member may be configured to move to (a) a blocking position to block the ultraviolet rays emitted by the ultraviolet unit from entering the main body, in conjunction with a positional movement of the dust bag, when the dust bag is detached from the main body, and (b) an open position so that the ultraviolet rays emitted by the ultraviolet unit enter the main body and the dust bag, in conjunction with a positional movement of the dust bag, when the dust bag is mounted on the main body.

### BRIEF DESCRIPTION OF DRAWINGS

FIG. 1 is a perspective view illustrating a cleaning device according to an embodiment.

FIG. 2 is a diagram illustrating a cross-sectional structure of the cleaning device of FIG. 1.

FIG. 3 is a perspective view illustrating a cleaner station of FIG. 1.

FIG. 4 is a diagram for describing a collection unit of the cleaner station of FIG. 3.

FIGS. 5A to 5C are diagrams for describing a structure for mounting a dust bag on the collection unit in FIG. 4.

FIG. 6 is a cross-sectional view of a station body of FIG. 5C, taken along line VI-VI.

FIG. 7 is a partial perspective view for describing a sterilization module of a cleaner station, according to an embodiment.

FIG. 8 is a cross-sectional view of a station body of FIG. 7, taken along line VIII-VIII.

FIG. 9 is a partial perspective view illustrating a state in which a dust bag is detached from the cleaner station of FIG. 7.

FIG. 10 is a perspective view for describing a sterilization module of a cleaner station, according to an embodiment.

FIGS. 11A and 11B are perspective views illustrating an assembled sterilization module according to an embodiment, seen from different angles.

FIG. 12 is an exploded perspective view of a sterilization module according to an embodiment.

FIG. 13 is a cross-sectional view illustrating an arrangement of a blocking member when a dust bag is mounted on a cleaner station, according to an embodiment.

FIG. 14 is a cross-sectional view illustrating an arrangement of a blocking member when a dust bag is detached from a cleaner station, according to an embodiment.

FIG. 15 is a diagram for describing a configuration for blocking an ultraviolet operation of a sterilization module when a frame cover is opened in a cleaner station, according to an embodiment.

FIG. 16 is a perspective view illustrating a vacuum cleaner according to an embodiment.

FIG. 17 is a block diagram schematically illustrating an internal configuration of a main body of the vacuum cleaner of FIG. 16.

### MODE FOR THE INVENTION

Hereinafter, example embodiments of the present disclosure will be described in detail with reference to the accompanying drawings. Like reference numerals in the drawings indicate parts or elements that perform substantially the same functions.

Terms such as “first” or “second” may be used to describe various elements, but the elements are not limited by the terms. These terms are only used to distinguish one element from another element. For example, a first element may be referred to as a second element, and a second element may be referred to as a first element in a similar manner, without departing from the scope of the present disclosure. The term “and/or” includes any and all combinations of one or more of the associated listed items.

Terms used herein are for describing embodiments and are not intended to limit the scope of the present disclosure. The singular expression also includes the plural meaning as long as it does not inconsistent with the context. In the present specification, it is to be understood that the terms such as “including,” “having,” and “comprising” are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added. Like reference numerals in the drawings indicate members that perform substantially the same functions.

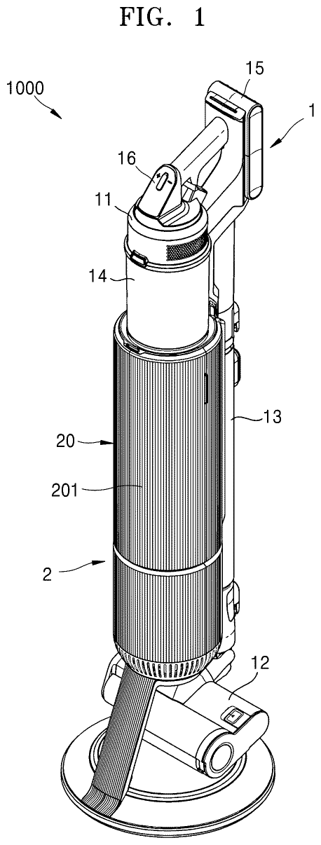

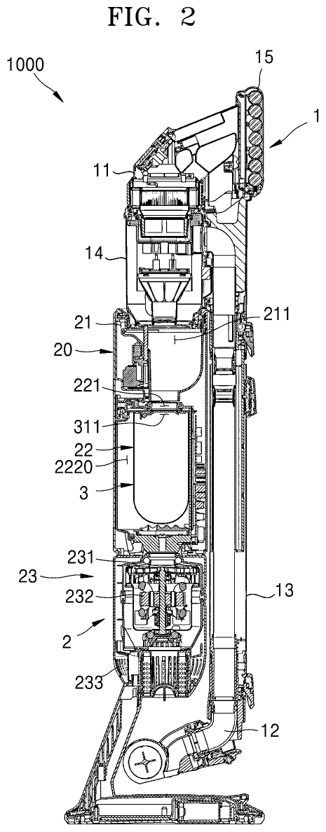

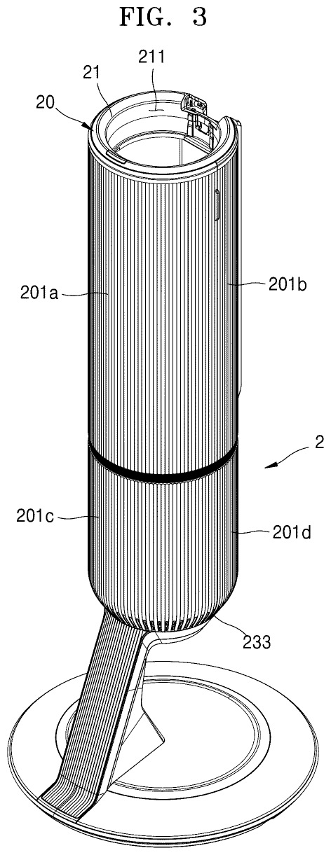

FIG. 1 is a perspective view illustrating a cleaning device 1000 according to an embodiment. FIG. 2 is a diagram illustrating a cross-sectional structure of the cleaning device 1000 of FIG. 1. FIG. 3 is a perspective view illustrating a cleaner station 2 of FIG. 1.

Referring to FIGS. 1 and 2, the cleaning device 1000 may include a vacuum cleaner 1, and the cleaner station 2 configured to be connected to the vacuum cleaner 1, and to remove foreign substances collected in the vacuum cleaner 1.

The vacuum cleaner 1 may include a cleaner body 11 and a dust collection container 14 detachably connected to the cleaner body 11. The dust collection container 14 may be provided to collect foreign substances that have been moved into the vacuum cleaner 1. The vacuum cleaner 1 may further include a suction pipe 13 detachably connected to the cleaner body 11, and a cleaner head 12 detachably connected to the suction pipe 13. Foreign substances, such as dust or hair, in air that is introduced through the vacuum cleaner head 12 may be collected in the dust collection container 14.

The vacuum cleaner body 11 may include a battery 15 provided to provide a driving force to the vacuum cleaner 1. The battery 15 may be detachably mounted on the vacuum cleaner body 11. The vacuum cleaner body 11 may include a manipulation unit 16. A user may turn the vacuum cleaner 1 on/off or adjust the suction strength by manipulating a power button, and the like provided on the manipulation unit 16.

Referring to FIGS. 2 and 3, the cleaner station 2 according to an embodiment is a device for removing or collecting foreign substances inside the dust collection container 14 of the vacuum cleaner 1, and may include a docking unit 21 to be connected to the dust collection container 14, a collection unit 22 on which a dust bag 3 for collecting foreign substances, and a suction unit 23 configured to provide a suction force to cause the foreign substances collected in the dust collection container 14 to be moved toward the dust bag 3. The cleaner station 2 includes a station body 20 on which the dust bag 3 is detachably mounted. The station body 20 may be referred to as a main body.

The station body 20 is configured to be connectable to the dust collection container 14 of the vacuum cleaner 1. For example, the docking unit 21 to be docked with the dust collection container 14 of the vacuum cleaner 1 may be provided in and connected to the station body 20. The docking unit 21 may be arranged in an upper portion of the station body 20. The docking unit 21 may provide a space in which the dust collection container 14 may be opened.

The station body 20 may have a substantially cylindrical shape. The station body 20 may include a housing 201 forming an exterior of the station body 20. For example, the housing 201 may include a first housing 201a, a second housing 201b, a third housing 201c, and a fourth housing 201d. The first housing 201a and the second housing 201b may form an upper exterior of the cleaner station 2, and the third housing 201c and the fourth housing 201d may form a lower exterior of the cleaner station 2.

The suction unit 23 configured to provide a suction force and the collection unit 22 on which the dust bag 3 is mounted may be provided inside the station body 20.

The suction unit 23 may generate a suction force such that foreign substances inside the vacuum cleaner 1 are discharged into the station body 20. As an example for this configuration, the suction unit 23 may include a suction fan 231 to generate an air flow, and a suction motor 232 to drive the suction fan 231. As the suction fan 231 rotates, air is introduced through a first opening 211 of the docking unit 21, and the introduced air passes through the dust bag 3 arranged in the collection unit 22 and is then discharged through a discharge port 233.

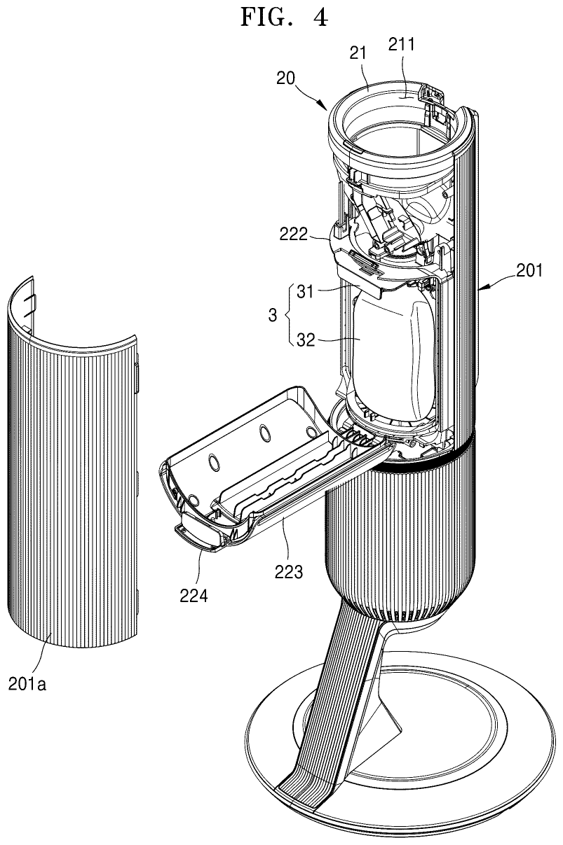

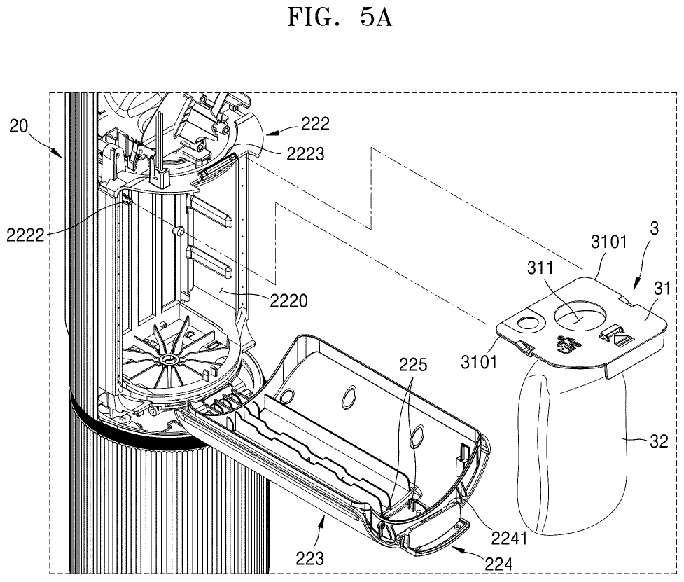

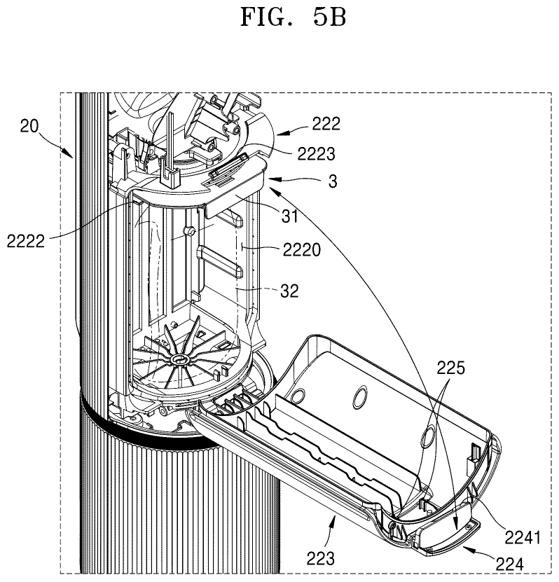

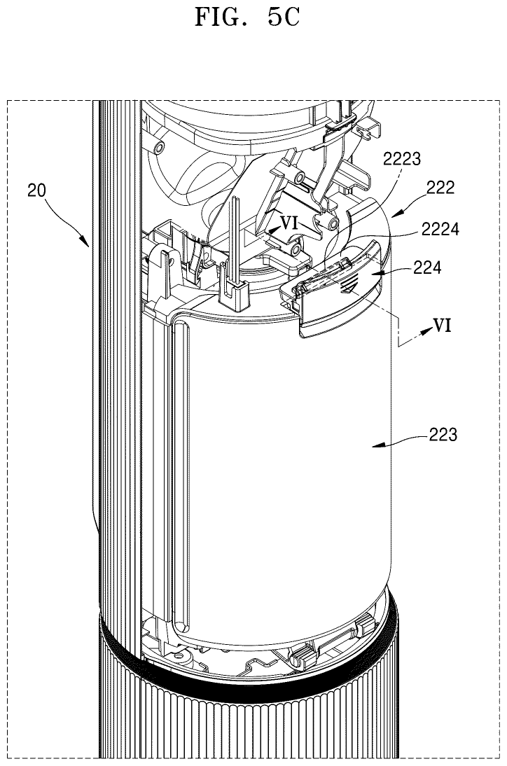

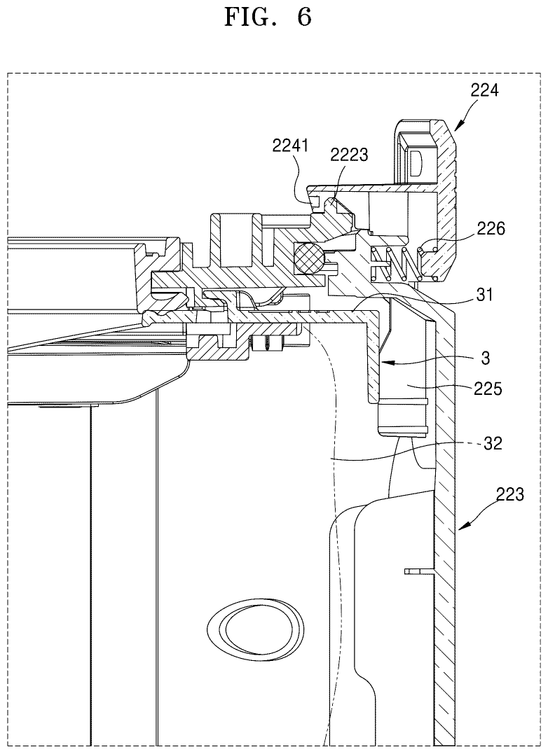

FIG. 4 is a diagram for describing the collection unit 22 of the cleaner station 2 of FIG. 3. FIGS. 5A to 5C are diagrams for describing a structure for mounting the dust bag 3 on the collection unit 22 in FIG. 4. FIG. 6 is a cross-sectional view of the station body of FIG. 5C.

Referring to FIGS. 2 and 4, in the station body 20, the first housing 201a may be separated from the other housings 201 to expose the collection unit 22. The collection unit 22 of the station body 20 includes a mounting frame 222 that provides a collection space 2220 in which the dust bag 3 is arranged, and a frame cover 223 installed in the mounting frame 222 to open and close the collection space 2220.

Referring to FIG. 2 and FIGS. 5A to 5C, the mounting frame 222 may be configured to have the dust bag 3 mounted thereon. For example, the mounting frame 222 may include coupling units 2222 to which the dust bag 3 is detachably coupled. The dust bag 3 may be slid to be coupled to the coupling units 2222 of the mounting frame 222. For example, both ends 3101 the dust bag 3 in a direction perpendicular to a mounting direction may be inserted and mounted into the coupling units 2222. When the dust bag 3 is pressed in the mounting direction, the dust bag 3 may be inserted into the coupling units 2222 of the mounting frame 222, and thus, the dust bag 3 may be mounted on the mounting frame 222. A second opening 221 through which the foreign substances discharged from the vacuum cleaner 1 may move is provided in the mounting frame 222. When the dust bag 3 is mounted, a foreign substance introduction port 311 of the dust bag 3 overlaps the second opening 221.

The frame cover 223 is configured to be openable. For example, the frame cover 223 may be configured to rotate about a certain axis. A locking unit 224 may be provided in the frame cover 223. The locking unit 224 may be arranged at the upper end of the frame cover 223. As the frame cover 223 rotates, the locking unit 224 may be coupled to the mounting frame 222.

Referring to FIGS. 5B, 5C, and 6, the locking unit 224 includes a locking hook 2241 to be coupled to the mounting frame 222. When closing the frame cover 223, the locking hook 2241 of the locking unit 224 is caught on a locking protrusion 2223. The locking unit 224 may be configured to be rotatable and may be pressed by an elastic member 226 to keep the locking hook 2241 caught on the locking protrusion 2223. Accordingly, the frame cover 223 may be kept closed by the locking unit 224. However, the opening and closing structure of the frame cover 223 is not limited thereto, and may be variously modified.

Pressing ribs 225 may be arranged on an inner surface of the frame cover 223. When closing the frame cover 223, the pressing ribs 225 come into contact with and press a mounting portion 31 of the dust bag 3. When the frame cover 223 is closed, the dust bag 3 may be kept mounted by the frame cover 223.

Referring again to FIG. 2, the cleaner station 2 according to the above-described embodiment may collect foreign substances inside the vacuum cleaner 1. For example, as the suction fan 231 of the suction unit 23 rotates, a suction force may be generated inside the station body 20. By the suction force, foreign substances inside the dust collection container 14 of the vacuum cleaner 1 may pass through the docking unit 21 to the collection unit 22 and be then collected into the dust bag 3. The foreign substances discharged from the vacuum cleaner 1 move with an air flow, pass through the second opening 221 and the foreign substance introduction port 311, and are collected in the dust bag 3, and the air excluding the foreign substances passes through the dust bag 3 and is then discharged through the discharge port 233.

The dust bag 3 in which the foreign substances are collected may be replaced. The dust bag 3 may be detachably coupled to the mounting frame 222. Accordingly, the used dust bag 3 may be detached and a new dust bag 3 may be assembled. For example, the used dust bag 3 may be detached in reverse order of mounting described above with reference to FIGS. 4, 5A to 5C, and FIG. 6. The user may detach part of the housing 201, for example, the first housing 201a, and then open the frame cover 223 of the collection unit 22. To open the frame cover 223, the user may pull an upper portion of the locking unit 224 to separate the locking hook 2241 of the locking unit 224 from the locking protrusion 2223 such that the frame cover 223 is opened. Then, when the mounting portion 31 of the existing dust bag 3 is pulled in a detachment direction, the dust bag 3 comes out from the coupling unit 2222 of the mounting frame 222, and thus, the dust bag 3 may be detached from the mounting frame 222. Thereafter, a new dust bag 3 may be mounted on the mounting frame 222.

However, until the dust bag 3 is replaced, the foreign substances may be stored in the dust bag 3. The longer the foreign substances are stored in the dust bag 3, the more likely it is that bacteria will grow in the foreign substances. The dust bag 3 mounted on the cleaner station 2 may have a large capacity. The capacity of the dust bag 3 may be greater than the capacity of the dust collection container 14 of the vacuum cleaner 1. For example, the capacity of the dust bag 3 may be 0.3 liters to 1.0 liters. As such, foreign substances may be stored in the large-capacity dust bag 3 for a long time, and may be vulnerable to hygiene issues such as bacterial growth.

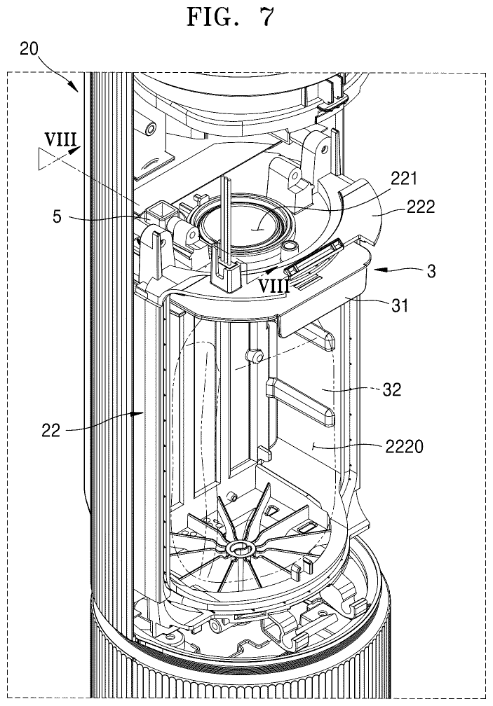

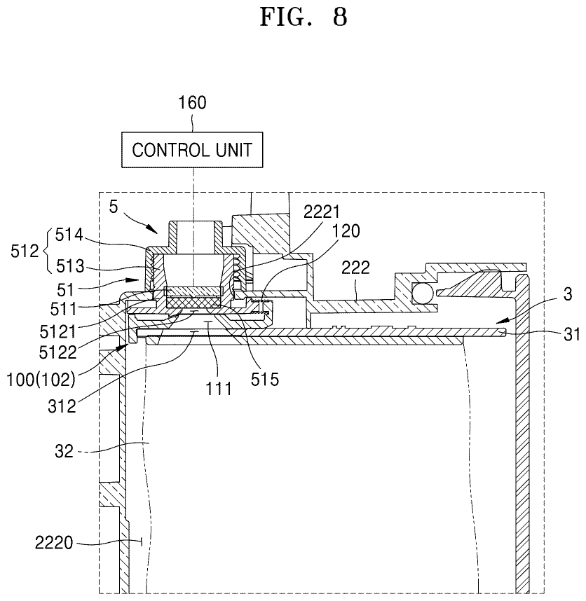

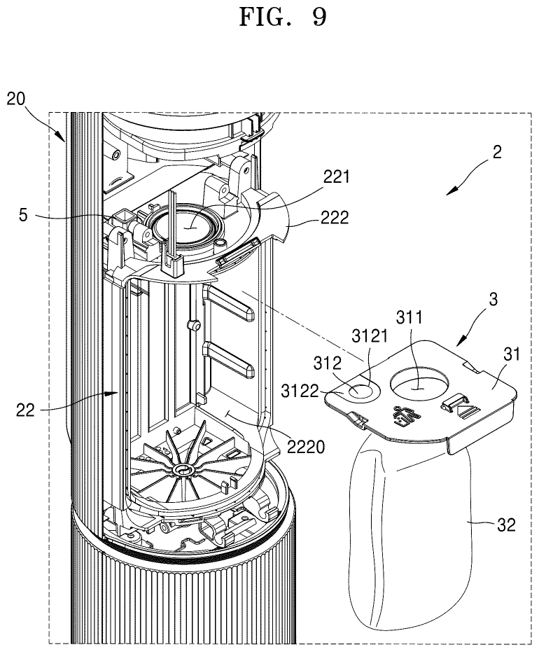

FIG. 7 is a partial perspective view for describing a sterilization module 5 of the cleaner station 2, according to an embodiment, and FIG. 8 is a cross-sectional view of the station body 20 of FIG. 7 taken along line VIII-VIII, for describing the sterilization module 5 according to an embodiment. FIG. 9 is a partial perspective view illustrating a state in which the dust bag 3 is detached from the cleaner station 2 of FIG. 7.

Referring to FIGS. 7 and 8, the cleaner station 2 according to an embodiment may further include the sterilization module 5 and a control unit 160 configured to control the operation of the sterilization module 5. The sterilization module 5 may be configured to sterilize the interior of the dust bag 3 mounted on the collection unit 22.

The sterilization module 5 may include an ultraviolet unit 51 configured to emit ultraviolet rays into the station body 20. The ultraviolet unit 51 may include an ultraviolet light source 511 configured to generate ultraviolet rays, and a light source support unit 512 to support the ultraviolet light source 511.

The ultraviolet light source 511 may generate ultraviolet rays having a wavelength that blocks bacterial growth in foreign substances. For example, the ultraviolet light source 511 may generate ultraviolet-C (UV-C). The ultraviolet light source 511 may generate light of a wavelength of 220 nm to 280 nm.

The light source support 512 may support the ultraviolet light source 511 such that the ultraviolet rays generated by the ultraviolet light source 511 are emitted into the station body 20, for example, into the collection space 2220. For example, the light source support unit 512 may include an edge portion 5121 surrounding the circumference of the ultraviolet light source 511, and an opening portion 5122 through which the ultraviolet rays generated by the ultraviolet light source 511 are emitted. A transparent member 515 that transmits ultraviolet rays may be arranged between the opening portion 5122 and the ultraviolet light source 511.

The light source support unit 512 may be arranged in the mounting frame 222. For example, the light source support unit 512 may include a first support unit 513 that supports the ultraviolet unit 51, and a second support unit 514 for fixing the first support unit 513 to the mounting frame 222. The first support unit 513 may have screw threads formed on the outer circumferential surface thereof, and the second support unit 514 may have screw threads formed on the inner circumferential surface thereof to engage with the screw threads of the first support unit 513. Accordingly, as the second support unit 514 is coupled to a portion of the first support unit 513 exposed upward after passing through an insertion hole 2221 of the mounting frame 222, the light source support unit 512 may be fixed to the mounting frame 222.

Referring to FIGS. 8 and 9, the dust bag 3 may include the mounting portion 31 and a bag portion 32 supported by the mounting portion 31. The mounting portion 31 may be configured to be detachably mounted on the mounting frame 222. The mounting portion 31 has a plate shape and may be coupled to the mounting frame 222 by sliding on the mounting frame 222. The mounting portion 31 includes the foreign substance introduction port 311 through which foreign substances are introduced.

The bag portion 32 may be configured to store therein foreign substances such as dust. The bag portion 32 may store foreign substances introduced through the foreign substance introduction port 311 of the mounting portion 31. The bag portion 32 may be made of a material that allows air to pass through but does not allow foreign substances in the air to pass through.

An ultraviolet window 312 that allows ultraviolet rays to pass therethrough may be arranged in the mounting portion 31. The ultraviolet window 312 may include an opening 3121 and a transparent film 3122 arranged on the opening 3121. Accordingly, ultraviolet rays generated by the ultraviolet light source 511 may be emitted into the dust bag 3 through the transparent film 3122.

When the dust bag 3 is mounted on the station body 20, the ultraviolet window 312 may overlap the ultraviolet light source 511. The ultraviolet window 312 may overlap the opening portion 5122 of the light source support unit 512. Accordingly, ultraviolet rays generated by the ultraviolet light source 511 may be emitted into the dust bag 3, for example, into the bag portion 32, through the opening portion 5122 of the light source support unit 512, and the ultraviolet window 312 of the dust bag 3.

Referring again to FIG. 8, ultraviolet rays generated by the ultraviolet unit 51 are emitted into the dust bag 3 through the ultraviolet window 312. The interior of the dust bag 3 may be sterilized by the ultraviolet rays emitted into the dust bag 3.

The control unit 160 may control the operation of the ultraviolet unit 51. For example, the control unit (160) may control the operation of the ultraviolet unit 51 such that the ultraviolet unit 51 operates periodically. For example, the control unit 160 may control the ultraviolet unit 51 to operate for a set period of time at regular intervals. For example, the ultraviolet unit 51 may be controlled to operate for 60 minutes to 120 minutes every 3 hours to 7 hours. For example, the ultraviolet unit 51 may be controlled to operate for 90 minutes every 5 hours.

However, when the ultraviolet rays generated by the ultraviolet unit 51 are exposed to the outside, they may cause damage to the user or other components. For example, when the user is irradiated with the ultraviolet rays while replacing the dust bag 3, the user may be harmed.

Taking this issue into consideration, the sterilization module 5 according to an embodiment may include a structure that physically blocks the ultraviolet unit 51 from being unintentionally exposed to the outside.

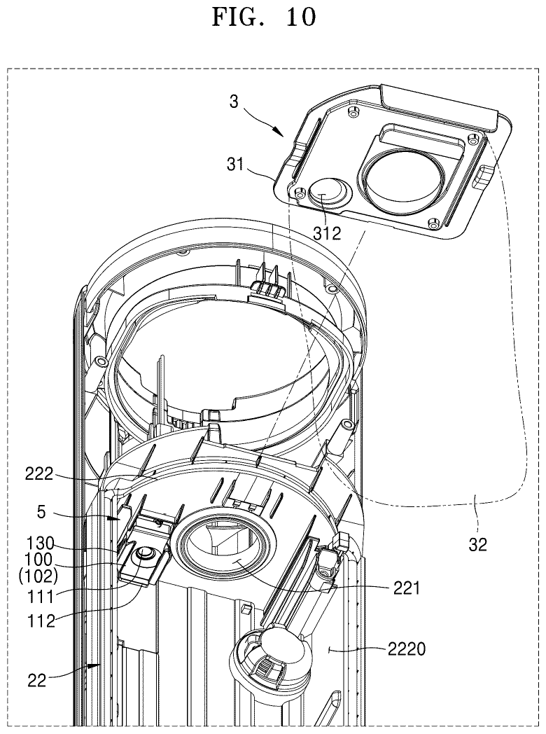

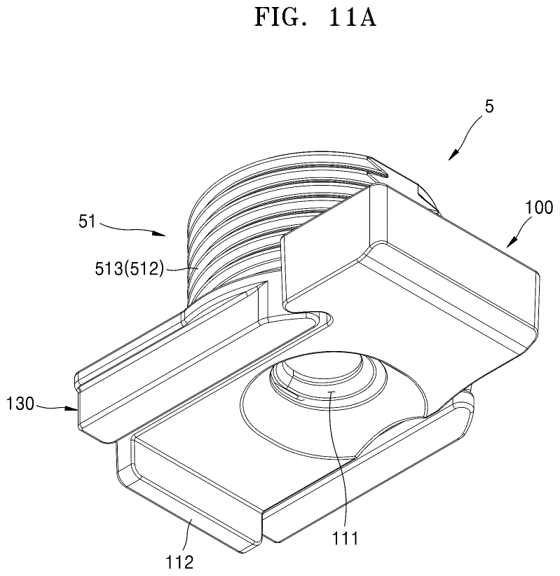

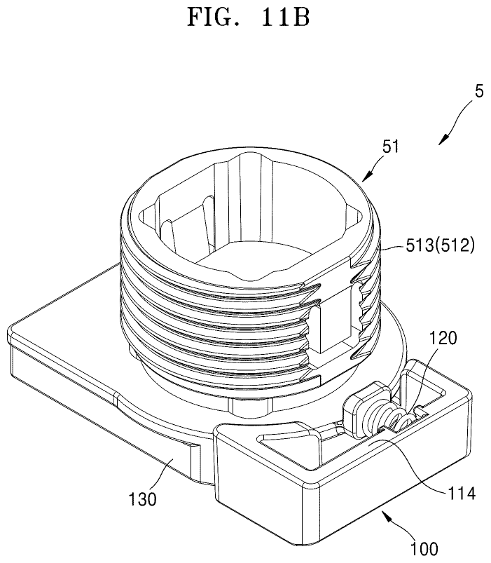

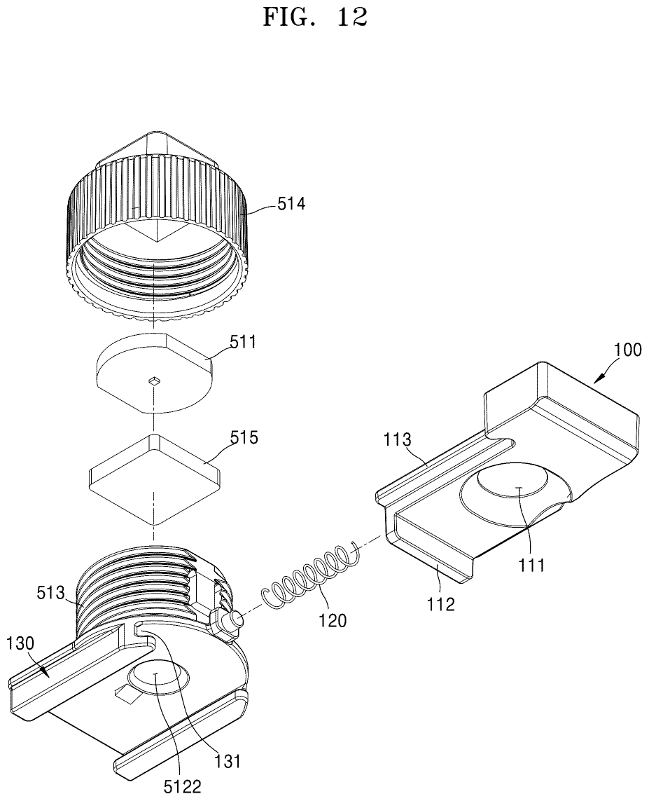

FIG. 10 is a perspective view for describing the sterilization module 5 of the cleaner station 2, according to an embodiment, and FIGS. 11A and 11B are perspective views illustrating the assembled sterilization module 5 according to an embodiment, seen from different angles. FIG. 12 is an exploded perspective view of the sterilization module 5 according to an embodiment.

Referring to FIGS. 10, 11A, 11B, and 12, the sterilization module 5 according to an embodiment may include a blocking member 100 that selectively blocks ultraviolet rays emitted by the ultraviolet unit 51.

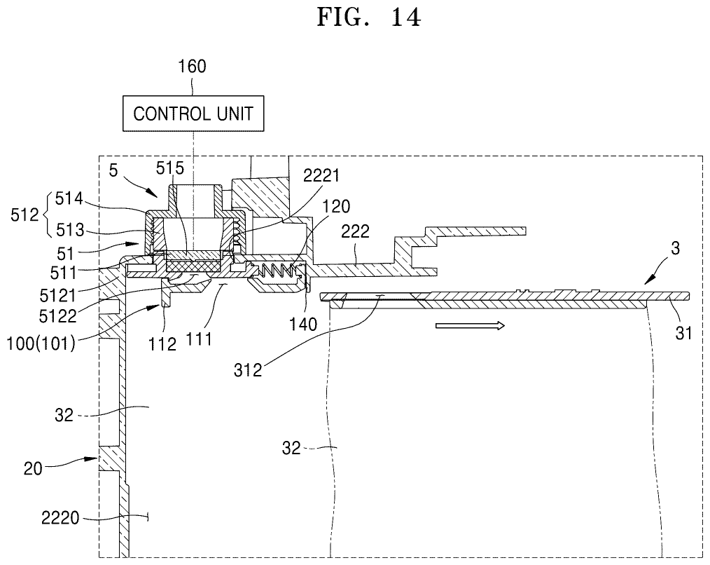

For example, the blocking member 100 may be movable in a direction perpendicular to the direction in which ultraviolet rays are emitted by the ultraviolet unit 51. The blocking member 100 may move in a direction parallel to the mounting and detaching directions of the dust bag 3. The blocking member 100 may move in the same direction as the mounting and detaching directions of the dust bag 3. The blocking member 100 may move between a blocking position 101 (see FIG. 14) to block ultraviolet rays emitted by the ultraviolet unit 51 from entering the station body 20, and an open position 102 to allow ultraviolet rays emitted by the ultraviolet unit 51 to enter the station body 20.

The blocking member 100 may be configured to move in conjunction with a positional movement of the dust bag 3 when the dust bag 3 is mounted on or detached from the station body 20. For example, when the dust bag 3 is detached from the station body 20, the blocking member 100 may move to the blocking position 101 to block ultraviolet rays emitted by the ultraviolet unit 51 from entering the station body 20, in conjunction with a positional movement of the dust bag 3. For example, when the dust bag 3 is mounted on the station body 20, the blocking member 100 may move to the open position 102 to allow ultraviolet rays emitted by the ultraviolet unit 51 to enter the station body 20, in conjunction with a positional movement of the dust bag 3.

For example, the blocking member 100 may include a contact area 112 that may come into contact with the dust bag 3. When the dust bag 3 is mounted, the contact area 112 may be pressed by the mounting portion 31 of the dust bag 3. Accordingly, when the dust bag 3 is mounted, the blocking member 100 may move in a direction parallel to the mounting direction.

The blocking member 100 may include an opening area 111 corresponding to the opening portion 5122 of the ultraviolet unit 51. When the blocking member 100 is at the open position 102, the opening area 111 may overlap the ultraviolet window 312 of the dust bag 3. When the blocking member 100 is at the open position 102, the ultraviolet unit 51 may overlap the opening area 111 of the blocking member 100. When the blocking member 100 is at the blocking position 101, the opening area 111 may not overlap the ultraviolet window 312. When the blocking member 100 is at the blocking position 101, the ultraviolet unit 51 may not overlap the opening area 111 of the blocking member 100. However, the opening area 111 of the blocking member 100 is an optional component, and may be omitted as needed.

For movement of the blocking member 100, the sterilization module 5 may further include an elastic member 120 that provides an elastic force to the blocking member 100, and a movement guide 130 that guides a movement of the blocking member 100.

The movement guide 130 may guide a movement of the blocking member 100 such that the blocking member 100 moves between the blocking position 101 and the open position 102. For example, the movement guide 130 may be configured to allow the blocking member 100 to slide. For example, the movement guide 130 may include a guide area 131 extending in a direction parallel to the mounting direction. The guide area 131 may have a groove shape into which a side portion 113 of the blocking member 100 may be inserted. However, the shape of the guide area 131 is not limited thereto and may vary depending on the shape of the blocking member 100. For example, although not illustrated, in a case in which the side portion 113 of the blocking member 100 has a groove shape, the guide area 131 may have a protrusion shape corresponding to the groove shape of the blocking member 100.

The elastic member 120 may provide an elastic force to press the blocking member 100 to move from the open position 102 to the blocking position 101. For example, the elastic member 120 may be arranged between the light source support unit 512 and one end 114 of the blocking member 100. The elastic member 120 may provide an elastic force in the direction in which the end 114 of the blocking member 100 moves away from the light source support unit 512.

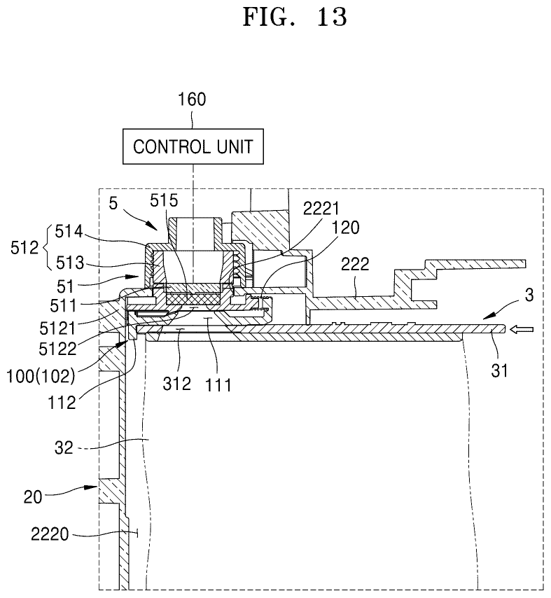

FIG. 13 is a cross-sectional view illustrating an arrangement of the blocking member 100 when the dust bag 3 is mounted on the cleaner station 2, according to an embodiment. FIG. 14 is a cross-sectional view illustrating an arrangement of the blocking member 100 when the dust bag 3 is detached from the cleaner station 2, according to an embodiment.

Referring to FIG. 13, the dust bag 3 may be mounted on the mounting frame 222 of the station body 20. For example, the dust bag 3 may be mounted in a first direction perpendicular to the direction in which ultraviolet rays are emitted, for example, in the left direction. When mounting the dust bag 3, the dust bag 3 may press the blocking member 100. For example, an end of the mounting portion 31 of the dust bag 3 may come into contact with the contact area 112 of the blocking member 100 to move the blocking member 100 in the same direction as the mounting direction. After the dust bag 3 is mounted on the mounting frame 222, the frame cover 223 may be closed to maintain the position of the dust bag 3. The dust bag 3 may be restricted from moving in the detachment direction by the pressing ribs 225 (see FIG. 6) provided on the inner surface of the frame cover 223, and may be fixed in position.

As the dust bag 3 and the blocking member 100 move in the mounting direction, the blocking member 100 may move from the blocking position 101 to the open position 102. Accordingly, the opening area 111 of the blocking member 100 overlaps the ultraviolet unit 51, and the ultraviolet window 312 of the dust bag 3 overlaps the ultraviolet unit 51. The ultraviolet unit 51 may be aligned with the ultraviolet window 312 and the opening area 111.

As the blocking member 100 is positioned at the open position 102, ultraviolet rays generated by the ultraviolet unit 51 may pass through the opening portion 5122, the opening area 111, and the ultraviolet window 312, and then enter the dust bag 3. The ultraviolet rays may enter the bag portion 32.

Referring to FIG. 14, the dust bag 3 may be detached from the mounting frame 222 in the detachment direction that is opposite to the mounting direction. For example, the dust bag 3 may be moved and detached in a second direction opposite to the first direction, for example, in the right direction. When detaching the dust bag 3, the blocking member 100 may be released from the pressure caused by the dust bag 3. The blocking member 100 may be moved in the same direction as the detachment direction of the dust bag 3, by the elastic force of the elastic member 120.

As the blocking member 100 moves in the detachment direction, the blocking member 100 may move from the open position 102 to the blocking position 101. The opening area 111 of the blocking member 100 does not overlap the ultraviolet unit 51, and the opening portion 5122 of the light source support unit 512 is blocked by the blocking member 100. Accordingly, ultraviolet rays emitted by the ultraviolet unit 51 are blocked by the blocking member 100 and thus do not enter the station body 20, for example, the collection space 2220.

The sterilization module 5 may further include a stopper 140 that limits the positional movement of the blocking member 100. The stopper 140 may cause the blocking member 100 to be stationary at the blocking position 101 after moving to the blocking position 101. Accordingly, when the dust bag 3 is detached, the blocking member 100 may be restricted by the stopper 140 from moving in the right direction, to be stationary at the blocking position 101.

As described above, the sterilization module 5 according to an embodiment may selectively block ultraviolet rays through a structure that is in mechanical conjunction with whether the dust bag 3 is detached. Accordingly, regardless of whether power is supplied to the ultraviolet unit 51, ultraviolet rays generated by the ultraviolet unit 51 may be blocked from being emitted toward the user, in mechanical conjunction with an operation of detaching the dust bag 3.

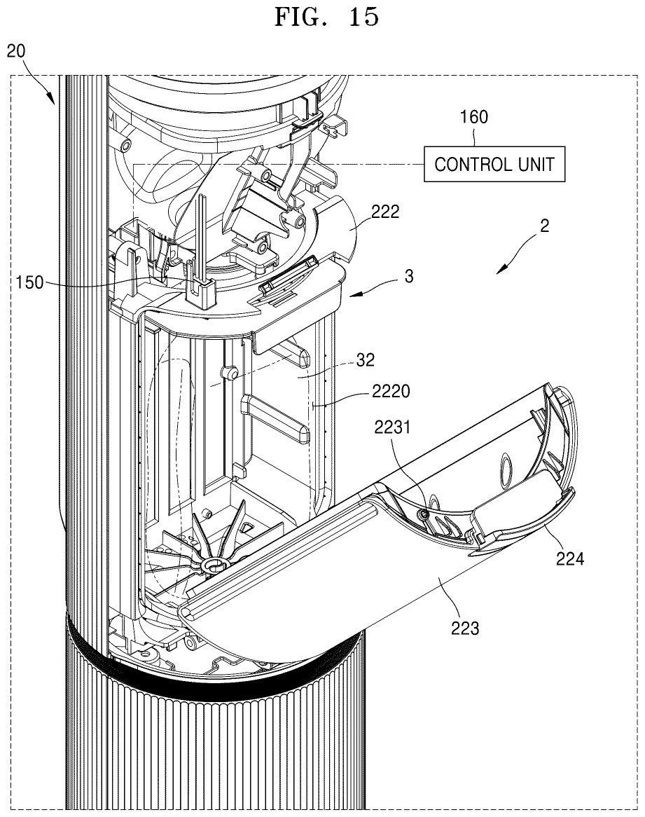

FIG. 15 is a diagram for describing a configuration for blocking an ultraviolet operation of the sterilization module 5 when the frame cover 223 is opened in the cleaner station 2, according to an embodiment.

Referring to FIG. 15, the cleaner station 2 may further include a component for selectively blocking an operation of the ultraviolet unit 51. For example, the cleaner station 2 may further include a component that selectively blocks an operation of the ultraviolet unit 51 in conjunction with an operation of the frame cover 223.

For example, the cleaner station 2 may further include a sensor member 150 configured to detect whether the frame cover 223 is opened or closed, and the control unit 160 configured to selectively stop an operation of the ultraviolet unit 51 based on a result of the detection by the sensor member 150.

The sensor member 150 may be configured to detect whether the mounting frame 222 is opened. For example, a magnet 2231 is installed in the frame cover 223, and the sensor member 150 may be a magnetic sensor configured to detect the magnet 2231 of the frame cover 223. For example, the sensor member 150 may detect a positional movement of the magnet 2231 to determine whether the frame cover 223 is opened.

The control unit 160 may stop or resume an operation of the ultraviolet unit 51 based on a result of the detection by the sensor member 150. When opening of the frame cover 223 is detected by the sensor member 150, the control unit 160 may stop the operation of the ultraviolet unit 51. When opening of the frame cover 223 is not detected by the sensor member 150, the control unit 160 may operate the ultraviolet unit 51.

In the cleaner station 2 according to an embodiment, with the configuration in which the operation of the ultraviolet unit 51 is stopped when the frame cover 223 is detached, damage caused by ultraviolet rays may be primarily prevented. In the cleaner station 2 according to an embodiment, even when the ultraviolet unit 51 is in operation due to an error in the sensor member 150, damage caused by ultraviolet rays may be prevented secondarily as the blocking member 100 moves in mechanical conjunction with an operation of detaching the dust bag 3 to block the ultraviolet rays. In other words, the cleaner station 2 according to an embodiment may reliably prevent damage from the ultraviolet rays by employing a double safety device to prevent damage due to ultraviolet rays.

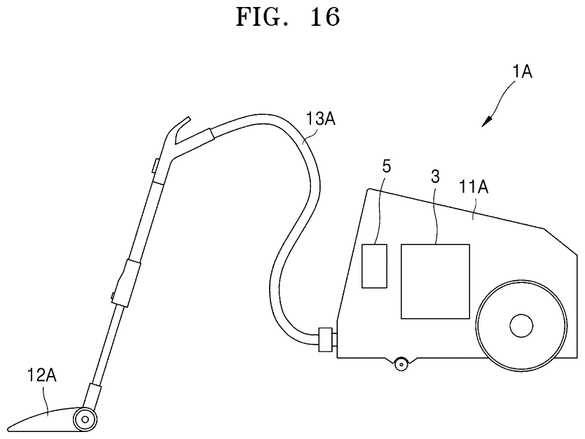

Meanwhile, the above embodiments have been described mainly with an example in which the sterilization module 5 is applied to the cleaner station 2, but the present disclosure is not limited thereto, and the sterilization module 5 may be applied to various devices as long as it is for sterilizing foreign substances in the dust bag 3. For example, the sterilization module 5 according to an embodiment may be applied to a vacuum cleaner 1A (see FIG. 16) on which the dust bag 3 is mounted.

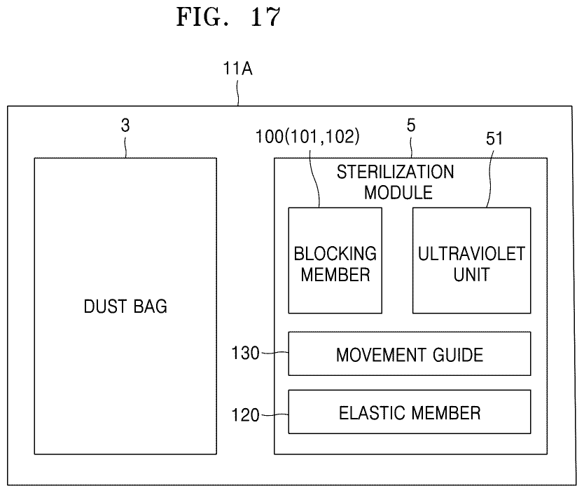

FIG. 16 is a perspective view illustrating the vacuum cleaner 1A according to an embodiment. FIG. 17 is a block diagram schematically illustrating an internal configuration of a cleaner body 11A of the vacuum cleaner 1A of FIG. 16. FIGS. 16 and 17 mainly illustrate the features of the disclosure, and the illustration of a suction unit and the like, which are the basic components of the vacuum cleaner 1A, is omitted.

Referring to FIGS. 16 and 17, the vacuum cleaner 1A according to an embodiment includes the cleaner body 11A, a suction head 12A provided to suck in foreign substances, and a connection portion 13A that connects the suction head 12A to the cleaner body 11A. The cleaner body 11A may be referred to as a main body.

The dust bag 3 is detachably mounted on the cleaner body 11A. The dust bag 3 collects foreign substances sucked in through the suction head 12A. The sterilization module 5 is arranged inside the cleaner body 11A. The sterilization module 5 may be configured to emit ultraviolet rays into the dust bag 3.

For example, the sterilization module 5 may include the ultraviolet unit 51 and the blocking member 100. The ultraviolet unit 51 emits ultraviolet rays into the cleaner body 11A. The ultraviolet unit 51 is the same as the ultraviolet unit 51 according to the above-described embodiments, and thus, redundant descriptions thereof will be omitted.

The blocking member 100 may selectively block the ultraviolet rays emitted by the ultraviolet unit 51. When the dust bag 3 is detached from the cleaner body 11A, the blocking member 100 may move to the blocking position 101 to block ultraviolet rays emitted by the ultraviolet unit 51 from entering the cleaner body 11A, in conjunction with a positional movement of the dust bag 3. When the dust bag 3 is mounted on the cleaner body 11A, the blocking member 100 may move to the open position 102 to allow ultraviolet rays emitted by the ultraviolet unit 51 to enter the cleaner body 11A, in conjunction with a positional movement of the dust bag 3. For movement of the blocking member 100, the sterilization module 5 may further include the movement guide 130 and the elastic member 120. The blocking member 100, the movement guide 130, and the elastic member 120 are the same as the blocking member 100, the movement guide 130, and the elastic member 120 according to the above-described embodiments, and thus, redundant descriptions thereof will be omitted.

Meanwhile, the above embodiments have been described mainly with an example of a structure in which the blocking member 100 slides in the same direction as the mounting and detachment directions of the dust bag 3, but the movement method and structure of the blocking member 100 are not limited thereto and may vary. Although not illustrated, the blocking member 100 may be configured to be rotatable about a certain axis.

For the purposes of promoting understanding of the disclosure, reference numerals are used in the preferred embodiments illustrated in the drawings, and particular terms are used to describe the embodiments, however, the disclosure is not limited by the terms and should be construed to encompass all components that would normally occur to those skilled in the art.

Particular executions described herein are merely examples and do not limit the scope of the disclosure in any way. For the sake of brevity, related-art electronics, control systems, software and other functional aspects of the systems may not be described in detail. Furthermore, line connections or connection members between elements depicted in the drawings represent functional connections and/or physical or circuit connections by way of example, and in actual applications, they may be replaced or embodied with various suitable additional functional connections, physical connections, or circuit connections. In addition, no item or component is essential to the practice of the disclosure unless the item or component is specifically described as being “essential” or “critical”. As used herein, the term such as “comprising”, “including” and the like are used to be understood as being an open-ended term for description.

The term ‘the’ and other demonstratives similar thereto in the specification of the disclosure (especially in the following claims) should be understood to include a singular form and plural forms. Furthermore, recitation of ranges of values herein are merely intended to serve as a shorthand method of referring individually to each separate value falling within the range, unless otherwise indicated herein, and each separate value is incorporated into the specification as if it were individually recited herein. Finally, the operations of the methods described herein may be performed in any suitable order unless otherwise indicated herein or otherwise clearly contradicted by context. The present disclosure is not limited to the described order of the operations. The use of any and all examples, or exemplary language (e.g., ‘and the like’) provided herein, is intended merely to better illuminate the present disclosure and does not pose a limitation on the scope of the present disclosure unless otherwise claimed. Furthermore, various changes and modifications will be readily apparent to one of ordinary skill in the art without departing from the spirit and scope of the disclosure.

According to an embodiment, a cleaner station and a cleaning device may be provided that ensure sterilization of a dust bag and user safety while reducing material costs, through a mechanical configuration for sterilizing the interior of the dust bag and selectively blocks ultraviolet rays in conjunction with an operation of detaching the dust bag from a main body.

A cleaner station according to an embodiment may include: a dust bag in which foreign substances are collected; a main body on which the dust bag is detachably mounted; and a sterilization module arranged inside the main body and configured to emit ultraviolet rays into the dust bag.

The sterilization module may include an ultraviolet unit configured to emit ultraviolet rays into the main body, and a blocking member to selectively block the ultraviolet rays emitted by the ultraviolet unit.

When the dust bag is detached from the main body, the blocking member may move to a blocking position to block the ultraviolet rays emitted by the ultraviolet unit from entering the main body, in conjunction with a positional movement of the dust bag.

When the dust bag is mounted on the main body, the blocking member may move to an open position to allow the ultraviolet rays emitted by the ultraviolet unit to enter the main body, in conjunction with a positional movement of the dust bag.

The sterilization module may further include a movement guide to guide the blocking member to be slidable, and an elastic member to provide an elastic force to cause the blocking member to move from the open position to the blocking position.

When the dust bag is detached from the main body, the blocking member may be released from pressure caused by the dust bag, and move from the open position to the blocking position due to the elastic force of the elastic member, and when the dust bag is mounted on the main body, the dust bag may press the blocking member to move the blocking member from the blocking position to the open position.

The sterilization module may further include a stopper to limit a positional movement of the blocking member.

The dust bag may include a mounting portion configured to be mounted on the main body, and a bag portion that is supported by the mounting portion and stores dust therein, and the mounting portion may include a foreign substance introduction port through which foreign substances are introduced, and an ultraviolet window to allow the ultraviolet rays to pass therethrough.

The ultraviolet unit may include an ultraviolet light source to generate ultraviolet rays, and a light source support unit to support the ultraviolet light source to face an interior of the main body, when the dust bag is mounted on the main body, the blocking member may be positioned at the open position, and the ultraviolet window overlaps the ultraviolet unit, and when the dust bag is detached from the main body, the blocking member is positioned at the blocking position, and the ultraviolet window may not overlap the ultraviolet unit.

The blocking member may move in directions that are identical to a mounting direction and a detachment direction of the dust bag.

The main body may include a mounting frame configured to provide a collection space in which the dust bag is arranged, and to have the dust bag mounted thereon, and a frame cover installed in the mounting frame to open and close the collection space.

The ultraviolet unit may be arranged to emit ultraviolet rays toward the collection space, and when the dust bag is detached from the mounting frame, the blocking member may move to the blocking position to block the ultraviolet rays from entering the collection space, in conjunction with a positional movement of the dust bag.

The cleaner station may further include a sensor member configured to detect whether the frame cover is opened or closed, and a control unit configured to selectively stop an operation of the ultraviolet unit based on a result of the detecting by the sensor member.

The cleaner station may further include a control unit configured to control an operation of the ultraviolet unit to operate periodically.

A capacity of the dust bag may be 0.3 liters to 1.0 liters.

A cleaning device according to an embodiment may include: a vacuum cleaner including a dust collection container in which foreign substances are collected; and a cleaner station including a docking unit connected to the dust collection container, a collection unit on which a dust bag to collect foreign substances is mounted, and a suction unit configured to provide a suction force to cause the foreign substances collected in the dust collection container to move toward the dust bag, wherein the cleaner station is configured to remove the foreign substances collected in the dust collection container.

The cleaner station may include: a main body on which the dust bag is detachably mounted; and a sterilization module arranged inside the main body and configured to emit ultraviolet rays into the dust bag.

The sterilization module may include an ultraviolet unit configured to emit ultraviolet rays into the main body, and a blocking member to selectively block the ultraviolet rays emitted by the ultraviolet unit.

The blocking member may move to a blocking position to block the ultraviolet rays emitted by the ultraviolet unit from entering the main body, in conjunction with a positional movement of the dust bag, when the dust bag is detached from the main body, and move to an open position to allow the ultraviolet rays emitted by the ultraviolet unit to enter the main body, in conjunction with a positional movement of the dust bag, when the dust bag is mounted on the main body.

The sterilization module may further include a movement guide to guide the blocking member to be slidable, and an elastic member to provide an elastic force to cause the blocking member to move from the open position to the blocking position.

A capacity of the dust bag may be greater than a capacity of the dust collection container.

1. A cleaner station comprising:
a main body on which a dust bag is detachably mountable to collect foreign substances in the dust bag; and
a sterilization module including:
an ultraviolet unit configured to emit ultraviolet rays into the main body, and
a blocking member configured to move to
a blocking position to block the ultraviolet rays emitted by the ultraviolet unit from entering the main body, in conjunction with a positional movement of the dust bag, when the dust bag is detached from the main body, and
an open position so that the ultraviolet rays emitted by the ultraviolet unit enter the main body and the dust bag, in conjunction with a positional movement of the dust bag, when the dust bag is mounted on the main body.

2. The cleaner station of claim 1, wherein the sterilization module further includes:
a movement guide to guide the blocking member to be slidable, and
an elastic member to provide an elastic force to cause the blocking member to slide from the open position to the blocking position when the dust bag is detached from the main body.

3. The cleaner station of claim 2, wherein the sterilization module is configured so that,
when the dust bag is detached from the main body, the blocking member is released from pressure caused by the dust bag, and moves from the open position to the blocking position due to the elastic force of the elastic member, and
when the dust bag is mounted on the main body, the blocking member is pressed by the dust bag so that the blocking member moves from the blocking position to the open position.

4. The cleaner station of claim 3, wherein the sterilization module further includes a stopper to limit a positional movement of the blocking member.

5. The cleaner station of claim 1, further comprising:
the dust bag,
wherein
the dust bag includes a mounting portion configured to be mounted on the main body, and a bag portion that is supported by the mounting portion and configured to store foreign substances therein, and
the mounting portion includes a foreign substance introduction port through which foreign substances are introduced into the bag portion, and an ultraviolet window to allow the ultraviolet rays to pass therethrough into the bag portion.

6. The cleaner station of claim 5, wherein
the ultraviolet unit includes an ultraviolet light source to generate ultraviolet rays, and a light source support unit to support the ultraviolet light source to face an interior of the main body, and
the sterilization module and the dust bag are configured so that,
when the dust bag is mounted on the main body, the blocking member is positioned at the open position, and the ultraviolet window overlaps the ultraviolet unit, and
when the dust bag is detached from the main body, the blocking member is positioned at the blocking position, and the ultraviolet window does not overlap the ultraviolet unit.

7. The cleaner station of claim 1, wherein the blocking member is configured to move in directions that are identical to a mounting direction and a detachment direction of the dust bag.

8. The cleaner station of claim 1, wherein the main body includes a mounting frame configured to provide a collection space in which the dust bag is arrangeable, and to which the dust bag is mountable, and a frame cover installed in the mounting frame to open and close the collection space.

9. The cleaner station of claim 8, wherein
the ultraviolet unit is configured to emit ultraviolet rays toward the collection space, and,
when the dust bag is detached from the mounting frame, the blocking member is configured to move to the blocking position to block the ultraviolet rays from entering the collection space, in conjunction with a positional movement of the dust bag.

10. The cleaner station of claim 8, further comprising:
a sensor member configured to detect whether the frame cover is opened or closed, and
a control unit configured to selectively stop an operation of the ultraviolet unit based on a detection result of the sensor member.

11. The cleaner station of claim 1, further comprising:
a control unit configured to control an operation of the ultraviolet unit so that the ultraviolet unit operates periodically.

12. The cleaner station of claim 1, wherein a capacity of the dust bag is 0.3 liters to 1.0 liters.

13. A cleaning device comprising:
a vacuum cleaner including a dust collection container in which foreign substances are collectable; and
a cleaner station including:
a docking unit to which the dust collection container is dockable,
a main body to which a dust bag is detachably mountable,
a suction unit configured to provide a suction force to, with the dust collection container docked to the docking unit and the dust bag mounted to the main body, cause foreign substances collected in the dust collection container to move to the dust bag, and
a sterilization module including:
an ultraviolet unit configured to emit ultraviolet rays into the main body, and
a blocking member configured to move to
a blocking position to block the ultraviolet rays emitted by the ultraviolet unit from entering the main body, in conjunction with a positional movement of the dust bag, when the dust bag is detached from the main body, and
an open position so that the ultraviolet rays emitted by the ultraviolet unit enter the main body and the dust bag, in conjunction with a positional movement of the dust bag, when the dust bag is mounted on the main body.

14. The cleaning device of claim 13, wherein the sterilization module further includes a movement guide to guide the blocking member to be slidable, and an elastic member to provide an elastic force to cause the blocking member to slide from the open position to the blocking position when the dust bag is detached from the main body.

15. The cleaning device of claim 13, wherein a capacity of the dust bag is greater than a capacity of the dust collection container.
